(19) United States
(12) Patent Application Publication
(10) Pub. No.: US 2022/0000197 A1
(43) Pub. Date: Jan. 6, 2022

(54) ADHESIVELY ATTACHED EYE MASKS

(71) Applicant: SYED SHAH, MANASSAS, VA (US)

(72) Inventor: SYED SHAH, MANASSAS, VA (US)

(21) Appl. No.: 17/367,533

(22) Filed: Jul. 5, 2021

Related U.S. Application Data
(60) Provisional application No. 63/161,922, filed on Mar. 16, 2021.
(60) Provisional application No. 63/122,628, filed on Dec. 8, 2020.
(60) Provisional application No. 63/048,647, filed on Jul. 6, 2020.

(51) Int. Cl. A41D 13/11; A61F 9/02

(52) U.S. Cl. CPC A41D 13/1184; A61F 9/029; A61F 9/026; A41D 13/1169

(57) ABSTRACT

An adhesively attached eye mask includes a transparent or semi-transparent plastic eyepiece for positioning over a user's eyes and an integral elongated transparent plastic temple or tab extending rearward from each side edge of the eyepiece for contacting the sides of a user's face. A peripheral edge of the eyepiece can include an adhesive. A face mask can be connected to a lower edge of the eye mask for covering the user's nose and mouth. A peripheral edge of the face mask can include an adhesive. A flexible loop or band can be positioned at each side of the face mask.

### CROSS-REFERENCE TO RELATED APPLICATION

This application claims the benefit of U.S. Provisional Patent Application No. 63/048,647, filed on Jul. 6, 2020, U.S. Provisional Patent Application No. 63/122,628, filed on Dec. 8, 2020, and U.S. Provisional Patent Application No. 63/161,922, filed on Mar. 16, 2021.

### BACKGROUND

### 1. Field

The disclosure of the present patent application relates to personal protection equipment (PPE), and particularly, to adhesively attached eye masks.

### 2. Description of the Related Art

In general, eye masks or other devices for protecting the eyes are attached to a user's face with straps or earpieces. These devices are typically bulky and heavy. In addition, conventional devices can interfere with movement of the user's head and become difficult. This contact reduces the strength of the connection and can result in pain from pulled hairs when the mask is removed. Should the wearer wish to have respiratory protection as well, a separate face mask is typically required.

Thus, adhesively attached eye masks solving the aforementioned problems are desired.

### SUMMARY

An embodiment of the adhesively attached eye mask includes a transparent or semitransparent (partially light blocking) plastic eyepiece for positioning over a user's eyes and an integral elongated transparent plastic temple or tab extending rearward from each side edge of the eyepiece for contacting the sides of a user's face. In an embodiment, a perimeter of the interior surface of the eye mask and an interior surface of the temple(s) that contact the user's skin can be coated with an adhesive material. The adhesive facilitates direct adhesion of the eye mask to the skin of the user. A paper release layer may be provided over the adhesive to protect the adhesive until the eye mask is in use. In an embodiment, an adhesive strip is positioned along a peripheral edge of the eyepiece, with a portion of the strip adhered to the edge of the eyepiece and a remaining portion of the strip extending beyond the edge the eyepiece, for contacting and sticking to the skin of the user. In an embodiment, an adhesive strip is provided along an inner surface of each temple. A paper release layer may be provided over the exposed adhesive of the strip or the adhesive on the inner mask surface to protect the adhesive until the eye mask is in use.

In an embodiment, a face mask can be connected to a lower edge of the eye mask for covering the user's nose and mouth. In an embodiment a peripheral edge of the inner surface of the face mask can be coated with an adhesive material. In an embodiment, an adhesive strip is positioned along a peripheral edge of the face mask, with a portion of the strip adhered to the edge of the face mask and a remaining portion of the strip extending beyond the edge of the face mask for contacting and sticking to the skin of the user. A flexible loop or band can be positioned at each side of the face mask.

The eye mask can be made of foldable and flexible plastic material, e.g. any material known to be suitable for sunglasses. The plastic material may be polycarbonate or Trivex (a urethane-based monomer produced by PPG), or a combination of both. In some embodiments, the plastic material may be mirrored or otherwise tinted so that others cannot see the eyes of the user. Due to the sealing nature of the adhesively attached eye mask, a user's eyes are protected from tiny droplets, air particulates, pollutants, and unwanted air and light.

A body of the face mask can be made of one or more layers of flexible sheet material, such as natural or synthetic cloth or other breathable and filtering materials that do not require compliance with medical regulations. The body of the face mask can be dimensioned and configured to cover the user's nose and mouth as a measure of protection against the spread of infectious disease that may be communicated by respiration through inhalation or exhalation of pathogens. A continuous release layer can be provided over the adhesive strip or the adhesive on the inner surface of the face mask, which can be removed by pulling one of two pull tabs on the sides of the release layer, until the release layer is completely removed from the face mask.

These and other features of the present subject matter will become readily apparent upon further review of the following specification.

### BRIEF DESCRIPTION OF THE DRAWINGS

FIG. 1 is an environmental perspective view of a first embodiment of an adhesively attached eye mask, showing the mask attached to a user's face.

FIG. 2 is an environmental perspective view of a second embodiment of an adhesively attached eye mask, showing the mask attached to a user's face.

FIG. 3 is a front perspective view of a third embodiment of an adhesively attached eye mask.

FIG. 4 is a rear perspective view of the adhesively attached eye mask of FIG. 3.

FIG. 5 is a front perspective view of a fourth embodiment of an adhesively attached eye mask.

FIG. 6 is a rear perspective view of the adhesively attached eye mask of FIG. 5.

FIG. 7 is an environmental perspective view of a fifth embodiment of an adhesively attached eye mask, showing the mask attached to a user's face.

FIG. 8 is an environmental perspective view of a sixth embodiment of an adhesively attached eye mask, showing the mask attached to a user's face.

FIG. 9 is an environmental, perspective view of a first embodiment of an adhesively attached eye mask with an integral face mask showing the mask attached to a user's face.

FIG. 10 is an environmental perspective view of a second embodiment of an adhesively attached eye mask with an integral face mask, showing the mask attached to a user's face.

FIG. 11 is an environmental perspective view of a third embodiment of an adhesively attached eye mask with an integral face mask, showing the mask attached to a user's face.

FIG. 12 is a rear perspective view of the adhesively attached eye mask with an integral face mask of FIG. 11.

FIG. 13 is an environmental perspective view of a fourth embodiment of an adhesively attached eye mask with an integral face mask, showing the mask attached to a user's face.

FIG. 14 is a rear perspective view of the adhesively attached eye mask with an integral face mask of FIG. 13.

FIG. 15 is an environmental perspective view of a fifth embodiment of an adhesively attached eye mask with an integral face mask, showing the mask attached to a user's face.

FIG. 16 is an environmental perspective view of a sixth embodiment of an adhesively attached eye mask with an integral face mask, showing the mask attached to a user's face.

FIG. 17 is an environmental, perspective view of a seventh embodiment of an adhesively attached eye mask, showing the mask attached to a user's face.

FIG. 18 is an environmental, perspective view of the seventh embodiment of an adhesively attached eye mask of FIG. 17, showing the release layer of the mask.

FIG. 19 is an environmental perspective view of a seventh embodiment of an adhesively attached eye mask with an integral face mask, showing the mask attached to a user's face.

FIG. 20 is a rear perspective view of the seventh embodiment of an adhesively attached eye mask with an integral face mask of FIG. 19, showing the release layer and tabs of the face mask.

FIG. 21 is an environmental perspective view of an eighth embodiment of an adhesively attached eye mask with an integral face mask, showing the mask attached to a user's face.

FIG. 22 is a rear perspective view of the eighth embodiment of an adhesively attached eye mask with an integral face mask of FIG. 21, showing the release layer and tabs of the face mask.

FIG. 23 is an environmental perspective view of a ninth embodiment of an adhesively attached eye mask with an integral face mask, showing the mask attached to a user's face.

FIG. 24 is a rear perspective view of the ninth embodiment of an adhesively attached eye mask with an integral face mask of FIG. 23, showing the release layer and tabs of the face mask.

FIG. 25 is an environmental perspective view of a tenth embodiment of an adhesively attached eye mask with an integral face mask, showing the mask attached to a user's face.

FIG. 26 is a rear perspective view of the tenth embodiment of an adhesively attached eye mask with an integral face mask of FIG. 25, showing the release layer and tabs of the face mask.

Similar reference characters denote corresponding features consistently throughout the attached drawings.

### DETAILED DESCRIPTION OF THE PREFERRED EMBODIMENTS

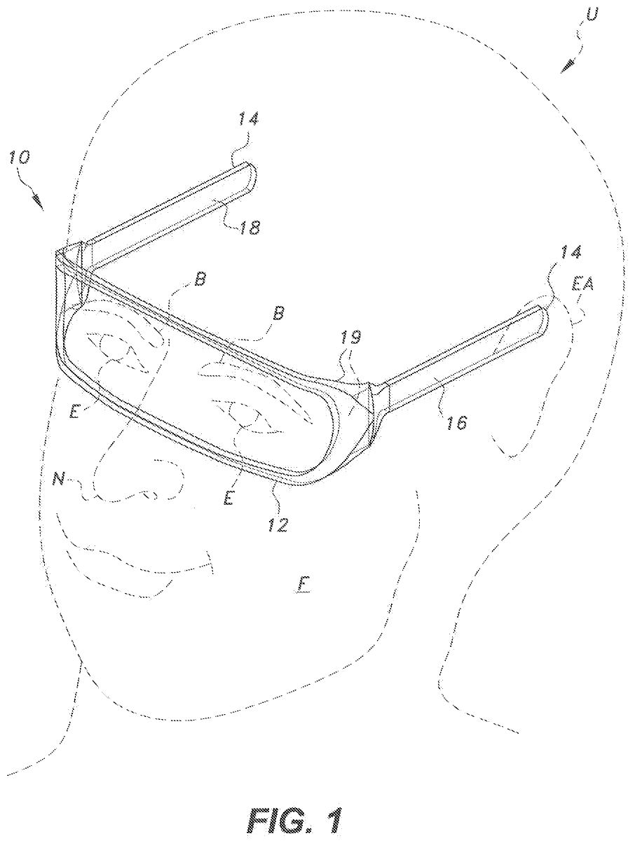

Adhesively attached eye masks and adhesively attached eye masks with integral face masks include a number of embodiments, as shown in FIGS. 1-18. The adhesively attached eye mask embodiments will be described first with respect to FIGS. 1-8 and 17-18. A first embodiment of the adhesively attached eye mask 10 is shown in FIG. 1, attached to the face F of a user U. The eye mask 10 includes a transparent (or partially light blocking) plastic eyepiece 12 having an inner peripheral surface 19 and two integral elongated transparent plastic temples or tabs 14 that are attached to the outside edges of the eyepiece 12 at their proximate ends and extend rearward therefrom. The two temples 14 include an external surface 16 away from the user's skin and an internal surface 18 adjacent to and in contact with the user's skin. The inner peripheral surface 19 of the eyepiece and the interior surface 18 of the two temples 14 that contact the user's skin are coated with an adhesive or sticky material. The adhesive provides a stronger grip by directly adhering the eyepiece 12 and the two temples 14 to the skin of the user. The eyepiece 12 may be tinted to provide UV protection in sunglass embodiments. In some embodiments, the eyepiece 12 may be mirrored or otherwise tinted so that others cannot see the eyes of the user.

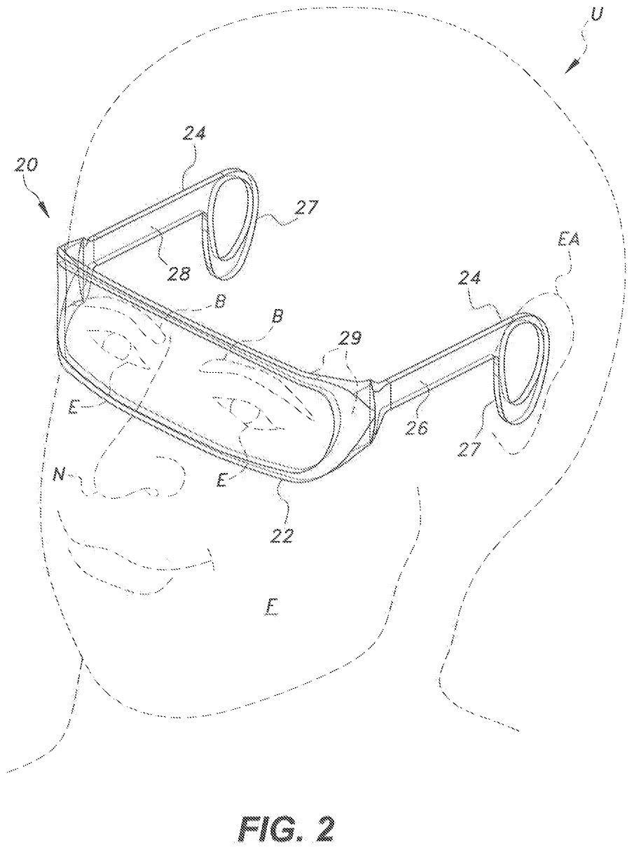

A second embodiment of the adhesively attached eye mask 20 is shown in FIG. 2, attached to the face F of a user U. As in the first embodiment, the eye mask 20 includes a transparent (or partially light blocking) plastic eyepiece 22 having an inner peripheral surface 29 and two integral elongated transparent plastic temples or tabs 24 that are attached to the outside edges of the eyepiece 22 at their proximate ends and extend rearward therefrom. The two temples 24 include an external surface 26 away from the user's skin and an internal surface 28 adjacent to and in contact with the user's skin. The inner peripheral surface 29 of the eyepiece and the interior surface 28 of the two temples 24 that contact the user's skin are coated with an adhesive or sticky material. The adhesive provides a stronger grip by directly adhering the eyepiece 22 and the two temples 24 to the skin of the user. The eyepiece 22 may be tinted for UV protection in sunglass embodiments. In some embodiments, the eyepiece 22 may be mirrored or otherwise tinted so that others cannot see the eyes of the user. In this embodiment, the adhesively attached eye mask 20 further includes an ear loop 27 that is attached to, and integral with, the distal end of each of the temples 24. The ear loops 27 provide additional support for the eye mask 20, by looping about the ears EA of the user U.

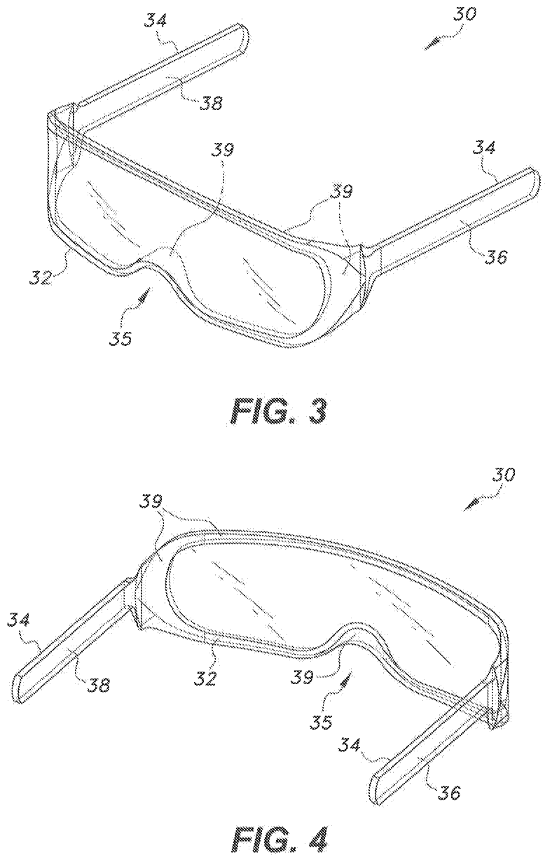

A third embodiment of the adhesively attached eye mask 30 is shown in FIGS. 3-4. As in the first embodiment, the eye mask 30 includes a transparent (or partially light blocking) plastic eyepiece 32 having an inner peripheral surface 39; and two integral elongated transparent plastic temples or tabs 34 that are attached to the outside edges of the eyepiece 32 at their proximate ends and extend rearward therefrom. The two temples 34 include an external surface 36 away from the user's skin and an internal surface 38 adjacent to and in contact with the user's skin. The inner peripheral surface 39 of the eyepiece and the interior surface 38 of the two temples 34 that contact the user's skin are coated with an adhesive or sticky material. The adhesive provides a stronger grip by directly adhering the eyepiece 32 and the two temples 34 to the skin of the user. The eyepiece 32 may be tinted for UV protection in sunglass embodiments. In some embodiments, the eyepiece 32 may be mirrored or otherwise tinted so that others cannot see the eyes of the user. In this embodiment, the eyepiece 32 includes a notch 35 along its bottom edge, to allow less of the user's nose N to be covered than in the first and second embodiments of the adhesively attached eye mask, if desired.

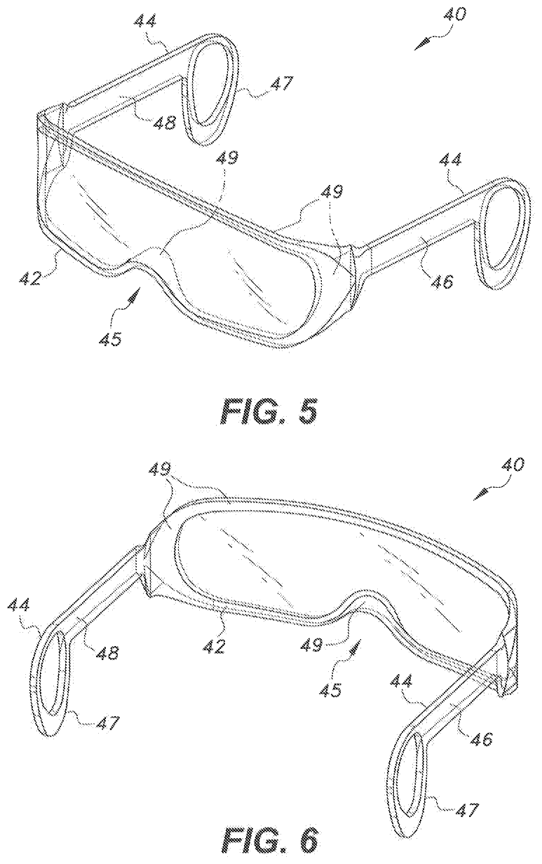

A fourth embodiment of the adhesively attached eye mask 40 is shown in FIGS. 5-6. As in the first embodiment, the eye mask 40 includes a transparent (or partially light blocking) plastic eyepiece 42 having an inner peripheral surface 49 and two integral elongated transparent plastic temples or tabs 44 that are attached to the outside edges of the eyepiece 42 at their proximate ends and extend rearward therefrom. The two temples 44 include an external surface 46 away from the user's skin and an internal surface 48 adjacent to and in contact with the user's skin. The inner peripheral surface 49 of the eyepiece and the interior surface 48 of the two temples 44 that contact the user's skin are coated with an adhesive or sticky material. The adhesive provides a stronger grip by directly adhering the eyepiece 42 and the two temples 44 to the skin of the user. The eyepiece 42 may be tinted for UV protection in sunglass embodiments. In some embodiments, the eyepiece 42 may be mirrored or otherwise tinted so that others cannot see the eyes of the user. In this embodiment, as in the third embodiment, the eyepiece 42 includes a notch 45 along its bottom edge, to allow less of the user's nose N to be covered than in the first and second embodiments of the adhesively attached eye mask, if desired.

Also, in this embodiment, as in the second embodiment 20, the adhesively attached eye mask 40 further includes an ear loop 47 that is attached to, and integral with, the distal end of each of the temples 44. The ear loops 47 provide additional support for the eye mask 40 by looping about the ears EA of the user U.

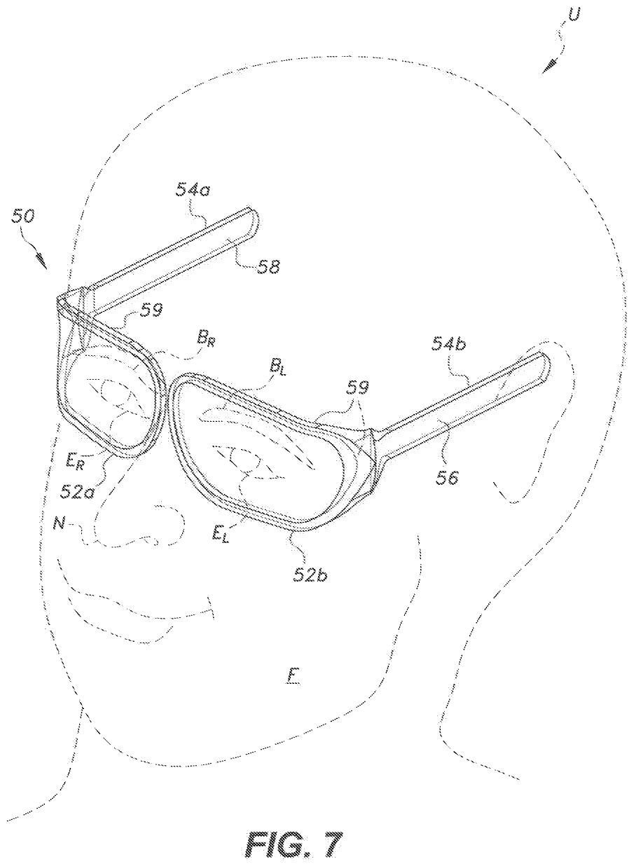

A fifth embodiment of the adhesively attached eye mask 50 is shown in FIG. 7. In this embodiment, the eye mask 50 includes two separate transparent (or partially light blocking) plastic eyepieces 52a, 52b for protecting the right eye ER and the left eye EL, respectively, of the user U. Each of the eyepieces 52a, 52b includes an inner peripheral surface 59 and an integral elongated transparent plastic temple or tab 54a, 54b that is attached to the outside edge of the corresponding eyepiece 52a, 52b at its proximate end and extends rearward therefrom. The two temples 54a, 54b include external surfaces 56 away from the user's skin and internal surfaces 58 adjacent to and in contact with the user's skin. The inner peripheral surfaces 59 of the eyepieces and the interior surfaces 58 of the two temples 54a, 54b that contact the user's skin are coated with an adhesive or sticky material. The adhesive provides a stronger grip by directly adhering the eyepieces 52a, 52b and the two temples 54a, 54b to the skin of the user. The eyepieces 52a, 52b may be tinted for UV protection in sunglass embodiments. In some embodiments, the eyepieces 52a, 52b may be mirrored or otherwise tinted so that others cannot see the eyes of the user. In this embodiment, the use of two separate eyepieces 52a, 52b allows most, if not all, of the user's nose N to remain uncovered.

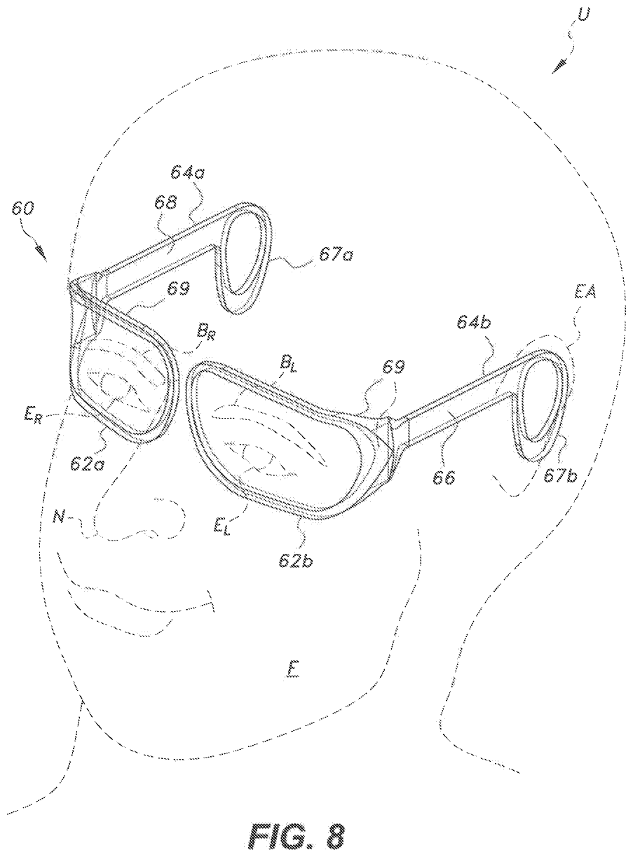

A sixth embodiment of the adhesively attached eye mask 60 is shown in FIG. 8. In this embodiment, as in the fifth embodiment, the eye mask 50 includes two separate transparent (or partially light blocking) plastic eyepieces 62a, 62b, for protecting the right eye ER and the left eye EL, respectively, of the user U. Each of the eyepieces 62a, 62b includes an inner peripheral surface 69; and an integral elongated transparent plastic temple or tab 64a, 64b that is attached to the outside edge of the corresponding eyepiece 62a, 62b at its proximate end and extends rearward therefrom. The two temples 64a, 64b include external surfaces 66 away from the user's skin and internal surfaces 68 adjacent to and in contact with the user's skin. The inner peripheral surfaces 69 of the eyepieces and the interior surfaces 68 of the two temples 64a, 64b that contact the user's skin are coated with an adhesive or sticky material. The adhesive provides a stronger grip by directly adhering the eyepieces 62a, 62b and the two temples 64a, 64b to the skin of the user. The eyepieces 62a, 62b may be tinted for UV protection in sunglass embodiments. In some embodiments, the eyepieces 62a, 62b may be mirrored or otherwise tinted so that others cannot see the eyes of the user. In this embodiment, the use of two separate eyepieces 62a, 62b allows most, if not all, of the user's nose N to remain uncovered. Also, in this embodiment, as in the third embodiment, the adhesively attached eye mask 60 further includes ear loops 67a, 67b that are attached to and integral with the distal ends of each of the temples 64a, 64b. The ear loops 67a, 67b provide additional support for the eye mask 60 by looping about the ears EA of the user U.

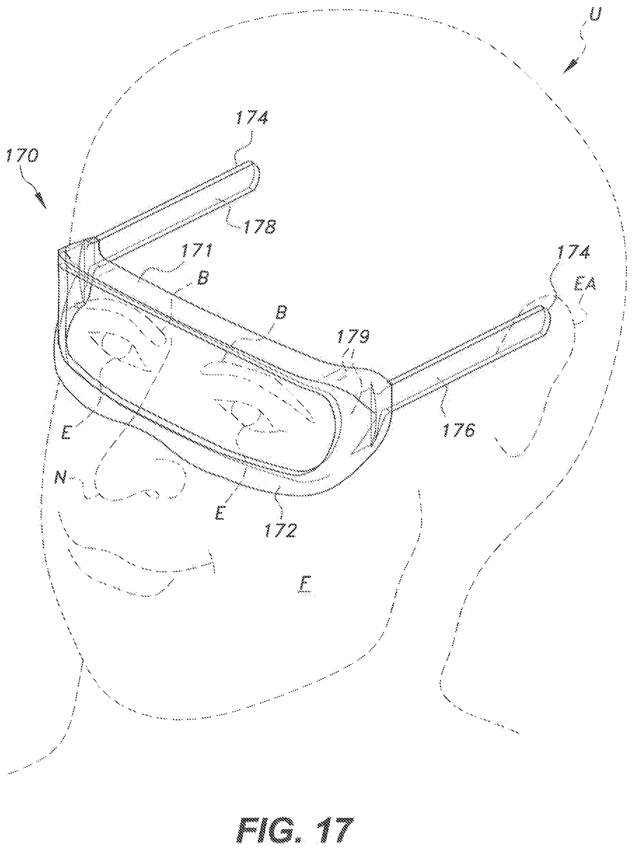

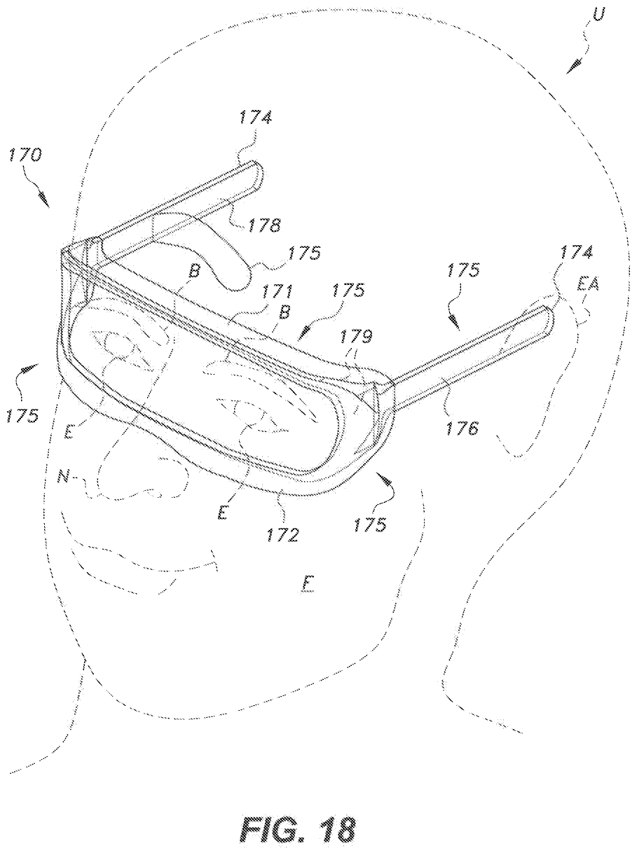

A seventh embodiment of the adhesively attached eye mask 170 is shown in FIGS. 17-18. As in the first embodiment, the adhesively attached eye mask 170 includes a transparent (or partially light blocking) plastic eyepiece 172 having an inner peripheral surface 179 and two integral elongated transparent plastic temples or tabs 174 that are attached to the outside edges of the eyepiece 172 at their proximate ends and extend rearward therefrom. The two temples 174 include an external surface 176 away from the user's skin and an internal surface 178 adjacent to and in contact with the user's skin. The eyepiece 172 may be tinted to provide UV protection in sunglass embodiments. In some embodiments, the eyepiece 172 may be mirrored or otherwise tinted so that others cannot see the eyes of the user. The adhesively attached eye mask 170 includes a transparent sealing tape 171 around its outside perimeter surface with a portion, e.g., half of the tape attached to the outside perimeter surface of the eyepiece 172, and a portion, e.g., half of the transparent sealing tape extending beyond the outside perimeter surface for attachment to the user's face F to create a seal so that smoke or other contaminants cannot get into the user's eyes. The tape 171 extends over the proximate end of the temple pieces 174. As is shown in FIG. 18, the exposed portion of the tape 171 and temple pieces 172 includes a continuous release layer 175 that is removed by peeling from the distal end of one of the temple pieces 174 until the release layer 175 is removed from the perimeter tape 171 and the other temple piece 174. It should be noted that the transparent sealing tape 171 can be used in any of the embodiments of the adhesively attached eye mask described herein. In addition, the release layer 175 can be used in any of the embodiments of the adhesively attached eye mask described herein, with or without the transparent sealing tape 171. The inner peripheral surface 179 of the eyepiece and the interior surface 178 of the two temples 174 that contact the user's skin can optionally be coated with an adhesive or sticky material to reinforce adhesion of the eyepiece 172 and the two temples 174 to the skin of the user.

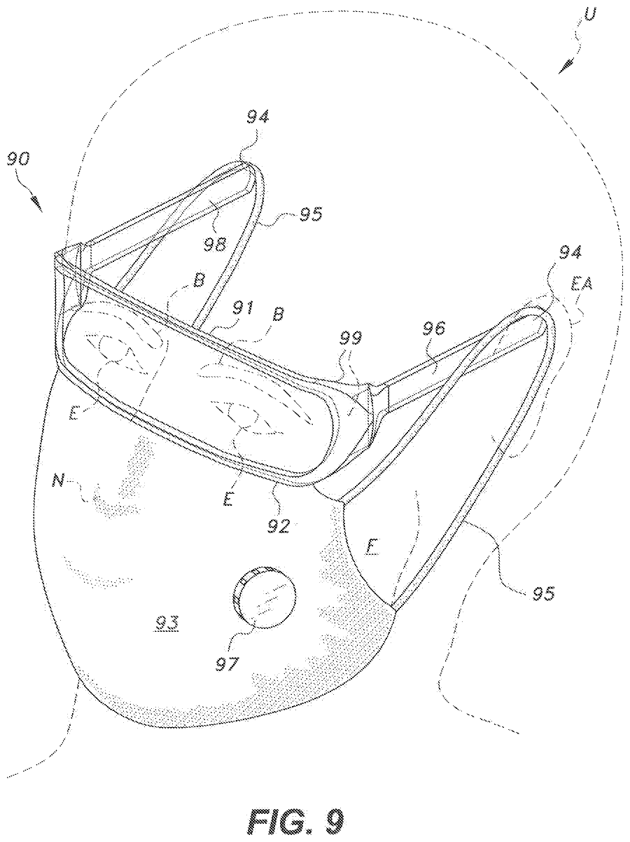

The adhesively attached eye mask with integral face mask includes a number of embodiments, as shown in FIGS. 9-16 and 19-26. A first embodiment of the adhesively attached eye mask with integral face mask, designated generally as 90, is shown in FIG. 9 attached to the face F of a user U. The adhesively attached eye mask 90 in this embodiment is similar to the adhesively attached eye mask 10 of FIG. 1, described above. The eye mask 90 includes a transparent (or partially light blocking) plastic eyepiece 91 having an inner peripheral surface 99 and two integral elongated transparent plastic temples or tabs 94 that are attached to the outside edges of the eyepiece 91 at their proximate ends and extend rearward therefrom. The two temples 94 include an external surface 96 away from the user's skin and an internal surface 98 adjacent to and in contact with the user's skin. The inner peripheral surface 99 of the eyepiece and the interior surface 98 of the two temples 94 that contact the user's skin are coated with an adhesive or sticky material. The adhesive provides a stronger grip by directly adhering the eyepiece 91 and the two temples 94 to the skin of the user. The eyepiece 91 may be tinted to provide UV protection in sunglass embodiments. In some embodiments, the eyepiece 91 may be mirrored or otherwise tinted so that others cannot see the eyes of the user. The integral face mask of the adhesively attached eye mask with integral face mask 90 includes a body 93, a top edge of which is attached to the bottom edge 92 of the eyepiece 91, and two ear loops 95 that are attached to the sides of the body 93 and extend rearward for attaching the face mask to the ears EA of the user U. The body 93 of the face mask 90 may optionally include a filter 97 or the face mask 90 may be without a filter.

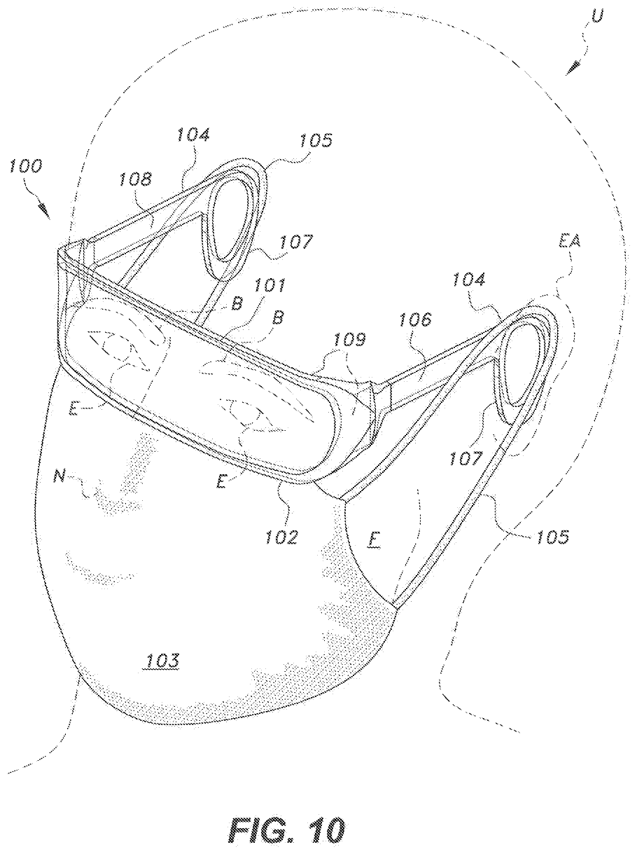

A second embodiment of the adhesively attached eye mask with integral face mask, designated generally as 100, is shown in FIG. 10 attached to the face F of a user U. The adhesively attached eye mask 100 in this embodiment is similar to the second embodiment of the adhesively attached eye mask 10 of FIG. 2, described above. The eye mask 100 includes a transparent (or partially light blocking) plastic eyepiece 101 having an inner peripheral surface 109 and two integral elongated transparent plastic temples or tabs 104 that are attached to the outside edges of the eyepiece 101 at their proximate ends and extend rearward therefrom. The two temples 104 include an external surface 106 away from the user's skin and an internal surface 108 adjacent to and in contact with the user's skin. The inner peripheral surface 109 of the eyepiece and the interior surface 108 of the two temples 104 that contact the user's skin are coated with an adhesive or sticky material. The adhesive provides a stronger grip by directly adhering the eyepiece 101 and the two temples 104 to the skin of the user. The eyepiece 101 may be tinted to provide UV protection in sunglass embodiments. In some embodiments, the eyepiece 101 may be mirrored or otherwise tinted so that others cannot see the eyes of the user. In this embodiment, the adhesively attached eye mask 100 further includes an ear loop 107 that is attached to, and integral with, the distal end of each of the temples 104. The ear loops 107 provide additional support for the eye mask 100, by looping about the ears EA of the user U. The integral face mask of the adhesively attached eye mask with integral face mask 100 includes a body 103, a top edge of which is attached to the bottom edge 102 of the eyepiece 101, and two ear loops 105 that are attached to the sides of the body 103 and extend rearward for attaching the face mask to the ears EA of the user U.

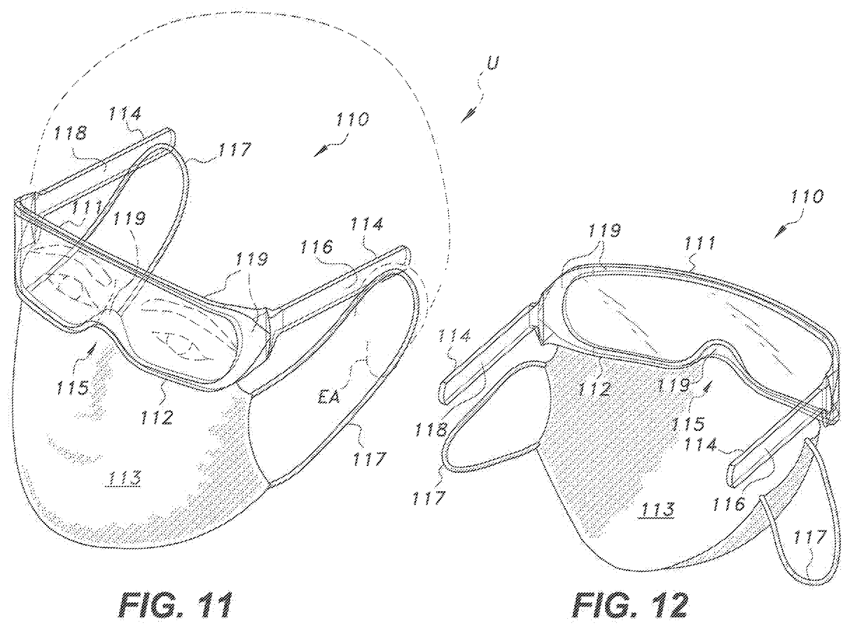

A third embodiment of the adhesively attached eye mask with integral face mask, designated generally as 110, is shown in FIGS. 11-12. The adhesively attached eye mask 110 in this embodiment is similar to the third embodiment of the adhesively attached eye mask 30 of FIGS. 3-4, described above. The eye mask 110 includes a transparent (or partially light blocking) plastic eyepiece 111 having an inner peripheral surface 119 and two integral elongated transparent plastic temples or tabs 114 that are attached to the outside edges of the eyepiece 111 at their proximate ends and extend rearward therefrom. The two temples 114 include an external surface 116 away from the user's skin and an internal surface 118 adjacent to and in contact with the user's skin. The inner peripheral surface 119 of the eyepiece and the interior surface 118 of the two temples 114 that contact the user's skin are coated with an adhesive or sticky material. The adhesive provides a stronger grip by directly adhering the eyepiece 111 and the two temples 114 to the skin of the user. In this embodiment, the eyepiece 111 includes a notch 115 along its bottom edge, to allow less of the user's nose to be covered by the eyepiece 111 than in some embodiments, if desired. The eyepiece 111 may be tinted to provide UV protection in sunglass embodiments. In some embodiments, the eyepiece 111 may be mirrored or otherwise tinted so that others cannot see the eyes of the user. The integral face mask of the adhesively attached eye mask with integral face mask 110 includes a body 113, a top edge of which is attached to the bottom edge 112 of the eyepiece 111, and two ear loops 117 that are attached to the sides of the body 113 and extend rearward for attaching the face mask to the ears EA of a user U.

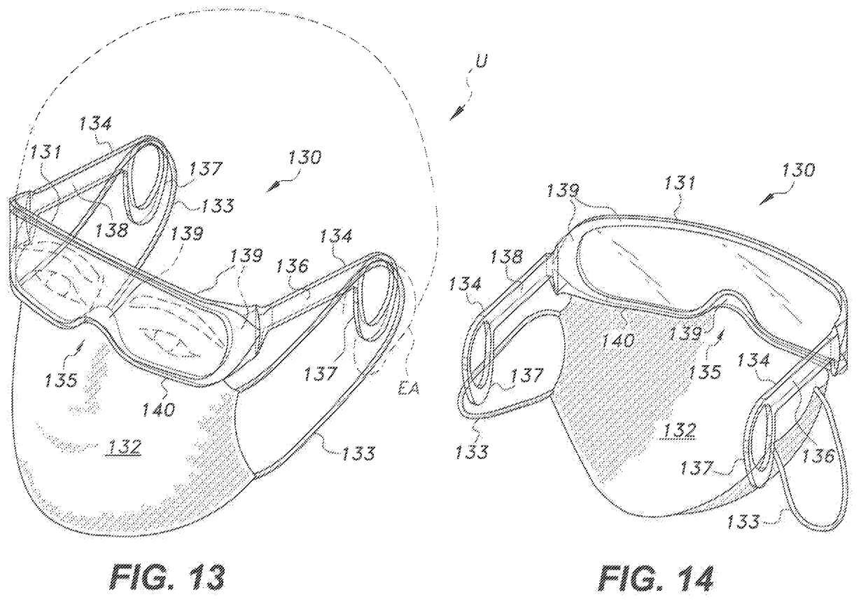

A fourth embodiment of the adhesively attached eye mask with integral face mask, designated generally as 130, is shown in FIGS. 13-14. The adhesively attached eye mask 130 in this embodiment is similar to the fourth embodiment of the adhesively attached eye mask 40 of FIGS. 5-6, described above. The eye mask 130 includes a transparent (or partially light blocking) plastic eyepiece 131 having an inner peripheral surface 139 and two integral elongated transparent plastic temples or tabs 134 that are attached to the outside edges of the eyepiece 131 at their proximate ends and extend rearward therefrom. The two temples 134 include an external surface 136 away from the user's skin and an internal surface 138 adjacent to and in contact with the user's skin. The inner peripheral surface 139 of the eyepiece and the interior surface 138 of the two temples 134 that contact the user's skin are coated with an adhesive or sticky material. The adhesive provides a stronger grip by directly adhering the eyepiece 131 and the two temples 134 to the skin of the user. In this embodiment, the eyepiece 131 includes a notch 135 along its bottom edge, to allow less of the user's nose to be covered by the eyepiece 131 than in some embodiments, if desired. The eyepiece 131 may be tinted to provide UV protection in sunglass embodiments. In some embodiments, the eyepiece 131 may be mirrored or otherwise tinted so that others cannot see the eyes of the user. The integral face mask of the adhesively attached eye mask with integral face mask 130 includes a body 132, the top of which is attached to the bottom edge 140 of the eyepiece 131, and two ear loops 133 that are attached to the sides of the body 132 and extend rearward for attaching the face mask to the ears EA of a user U.

Also, in this embodiment, as in the second embodiment 100, the adhesively attached eye mask 130 further includes an ear loop 137 that is attached to, and integral with, the distal end of each of the temples 134. The ear loops 137 provide additional support for the eye mask 130 by looping about the ears EA of the user U.

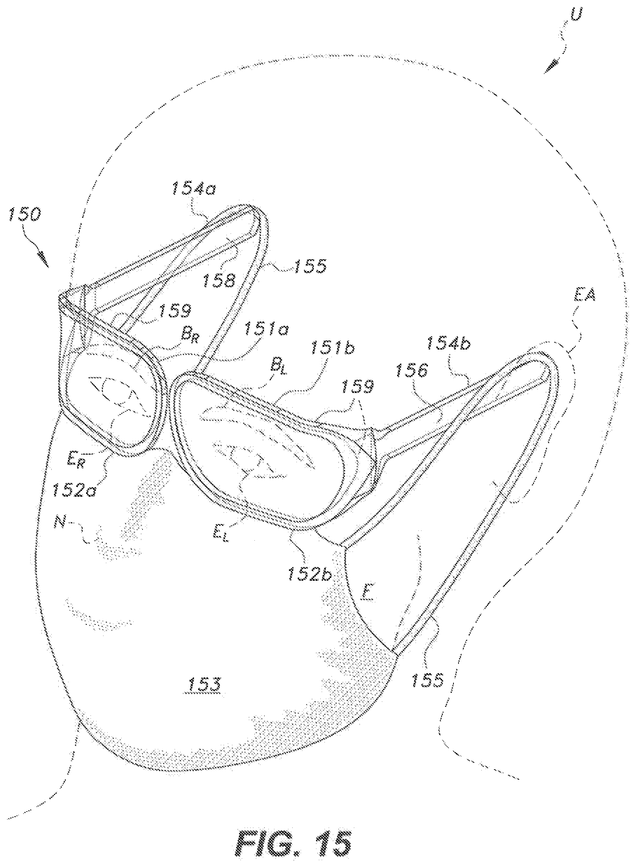

A fifth embodiment of the adhesively attached eye mask with integral face mask, designated generally as 150, is shown in FIG. 15. The adhesively attached eye mask 150 in this embodiment includes two separate transparent (or partially light blocking) plastic eyepieces 151a, 151b, for protecting the right eye ER and the left eye EL, respectively, of the user U. Each of the eyepieces 151a, 151b includes an inner peripheral surface 159 and an integral elongated transparent plastic temple or tab 154a, 154b that is attached to the outside edge of the corresponding eyepiece 151a, 151b at its proximate end and extends rearward therefrom. The two temples 154a, 154b include external surfaces 156 away from the user's skin and internal surfaces 158 adjacent to and in contact with the user's skin. The inner peripheral surfaces 159 of the eyepieces and the interior surfaces 158 of the two temples 151a, 151b that contact the user's skin are coated with an adhesive or sticky material. The adhesive provides a stronger grip by directly adhering the eyepieces 151a, 151b and the two temples 154a, 154b to the skin of the user. The eyepieces 151a, 151b may be tinted for UV protection in sunglass embodiments. In some embodiments, the eyepieces 151a, 151b may be mirrored or otherwise tinted so that others cannot see the eyes of the user. In this embodiment, the use of two separate eyepieces 151a, 151b allows most, if not all, of the user's nose N to remain uncovered. The integral face mask of the adhesively attached eye mask with integral face mask 150 includes a body 153, a top edge of which is attached to the bottom edges 152a, 152b of the eyepieces 151a, 151b, and two ear loops 155 that are attached to the sides of the body 153 and extend rearward for attaching the face mask to the ears EA of a user U.

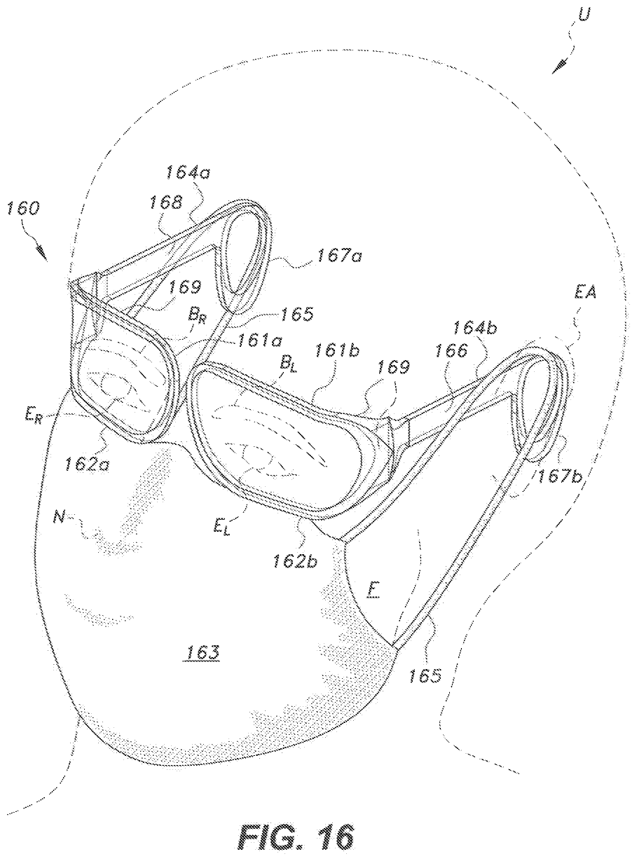

A sixth embodiment of the adhesively attached eye mask with integral face mask, designated generally as 160, is shown in FIG. 16. The adhesively attached eye mask 160 in this embodiment includes two separate transparent (or partially light blocking) plastic eyepieces 161a, 161b, for protecting the right eye ER and the left eye EL, respectively, of the user U. Each of the eyepieces 161a, 161b includes an inner peripheral surface 169; and an integral elongated transparent plastic temple or tab 164a, 164b that is attached to the outside edge of the corresponding eyepiece 161a, 161b at its proximate end and extends rearward therefrom. The two temples 164a, 164b include external surfaces 166 away from the user's skin and internal surfaces 168 adjacent to and in contact with the user's skin. The inner peripheral surfaces 169 of the eyepieces 161a, 161b and the interior surfaces 168 of the two temples 164a, 164b that contact the user's skin are coated with an adhesive or sticky material. The adhesive provides a stronger grip by directly adhering the eyepieces 161a, 161b and the two temples 164a, 164b to the skin of the user. The eyepieces 161a, 161b may be tinted for UV protection in sunglass embodiments. In some embodiments, the eyepieces 161a, 161b may be mirrored or otherwise tinted so that others cannot see the eyes of the user. In this embodiment, the use of two separate eyepieces 161a, 161b allows most, if not all, of the user's nose N to remain uncovered. Also, in this embodiment, as in previous embodiments, the adhesively attached eye mask 160 further includes ear loops 167a, 167b that are attached to, and integral with, the distal ends of each of the temples 164a, 164b. The ear loops 167a, 167b provide additional support for the eye mask 60 by looping about the ears EA of the user U. The integral face mask of the adhesively attached eye mask with integral face mask 160 includes a body 163, the top of which is attached to the bottom edges 162a, 162b of the eyepieces 161a, 161b, and two ear loops 165 that are attached to the sides of the body 163 and extend rearward for attaching the face mask to the ears EA of a user U.

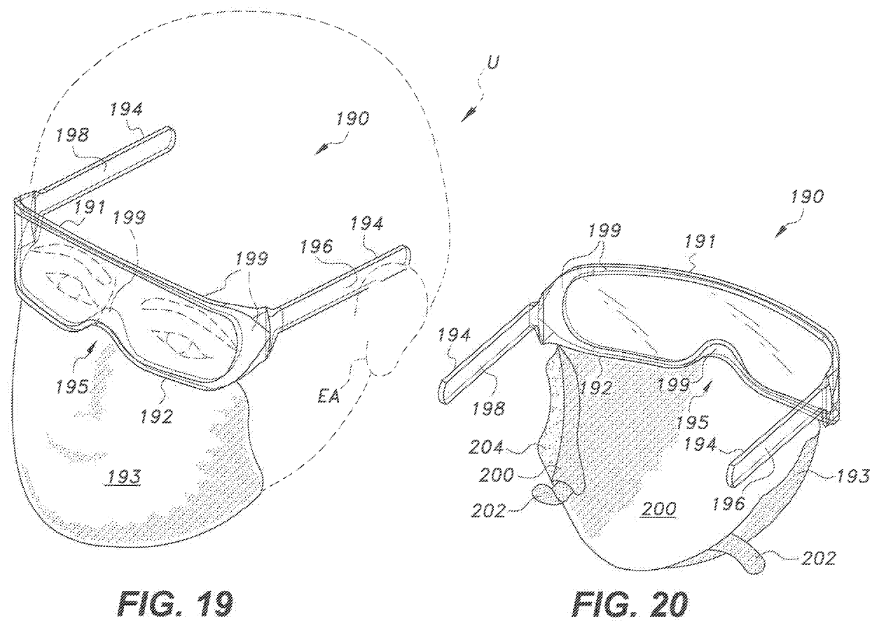

A seventh embodiment of the adhesively attached eye mask with integral face mask, designated generally as 190, is shown in FIGS. 19-20. The eye mask 190 includes a transparent (or partially light blocking) plastic eyepiece 191 having an inner peripheral surface 199 and two integral elongated transparent plastic temples or tabs 194 that are attached to the outside edges of the eyepiece 191 at their proximate ends and extend rearward therefrom. The two temples 194 include an external surface 196 away from the user's skin and an internal surface 198 adjacent to and in contact with the user's skin. The inner peripheral surface 199 of the eyepiece and the interior surface 198 of the two temples 194 that contact the user's skin are coated with an adhesive or sticky material. The adhesive provides a stronger grip by directly adhering the eyepiece 191 and the two temples 194 to the skin of the user. In this embodiment, the eyepiece 191 can include a notch 195 along its bottom edge, to allow less of the user's nose to be covered by the eyepiece 191 than in some embodiments, if desired. The eyepiece 191 may be tinted to provide UV protection in sunglass embodiments. In some embodiments, the eyepiece 191 may be mirrored or otherwise tinted so that others cannot see the eyes of the user. The integral face mask of the adhesively attached eye mask with integral face mask 190 includes a mask body 193, a top edge of which is attached to the bottom edge 192 of the eyepiece 191. In this embodiment, the interior perimeter of the mask body 193, includes an adhesive material 204 that is covered by a release layer 200. Pull tabs 202 are provided to allow a user to remove the release layer 200 prior to attaching the adhesively attached eye mask with integral face mask 190 to their face. The adhesive material 204 and the adhesive material on the inner peripheral surface 199 of the eyepiece create a seal to block liquid droplets, dust, and smoke and other air pollutants from the eyes, nose, and mouth of the user U.

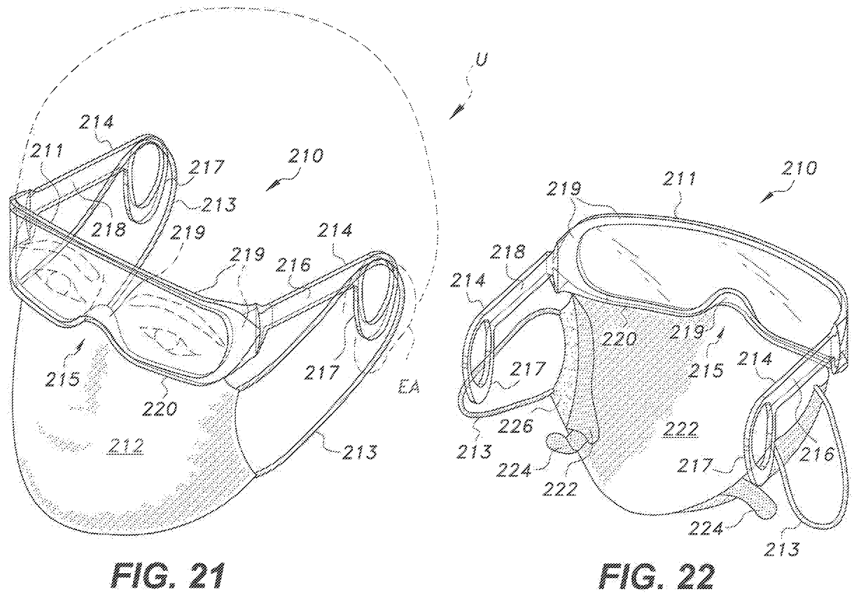

An eighth embodiment of the adhesively attached eye mask with integral face mask, designated generally as 210, is shown in FIGS. 21-22. The adhesively attached eye mask 210 in this embodiment is similar to the fourth embodiment of the adhesively attached eye mask 40 of FIGS. 5-6, described above. The eye mask 210 includes a transparent (or partially light blocking) plastic eyepiece 211 having an inner peripheral surface 219 and two integral elongated transparent plastic temples or tabs 214 that are attached to the outside edges of the eyepiece 211 at their proximate ends and extend rearward therefrom. The two temples 214 include an external surface 216 away from the user's skin and an internal surface 218 adjacent to and in contact with the user's skin. The inner peripheral surface 219 of the eyepiece and the interior surface 218 of the two temples 214 that contact the user's skin are coated with an adhesive or sticky material. The adhesive provides a stronger grip by directly adhering the eyepiece 211 and the two temples 214 to the skin of the user. In this embodiment, the eyepiece 211 includes a notch 215 along its bottom edge, to allow less of the user's nose to be covered by the eyepiece 231 than in some embodiments, if desired. The eyepiece 211 may be tinted to provide UV protection in sunglass embodiments. In some embodiments, the eyepiece 211 may be mirrored or otherwise tinted so that others cannot see the eyes of the user. The integral face mask of the adhesively attached eye mask with integral face mask 210 includes a body 212, the top of which is attached to the bottom edge 220 of the eyepiece 211, and two ear loops 213 that are attached to the sides of the body 212 and extend rearward for attaching the face mask to the ears EA of a user U.

Also, in this embodiment, as in the second embodiment 20, the adhesively attached eye mask 210 further includes an ear loop 217 that is attached to, and integral with, the distal end of each of the temples 214. The ear loops 217 provide additional support for the eye mask 210 by looping about the ears EA of the user U.

Also, in this embodiment, as in the seventh embodiment the interior perimeter of the mask body 219, includes an adhesive material 226 that is covered by a release layer 222. Pull tabs 224 are provided to allow a user to remove the release layer 222 prior to attaching the adhesively attached eye mask with integral face mask 210 to the face. The adhesive material 226 and the adhesive material on the inner peripheral surface 219 of the eyepiece create a seal to block liquid droplets, dust, and smoke and other air pollutants from the eyes, nose, and mouth of the user U.

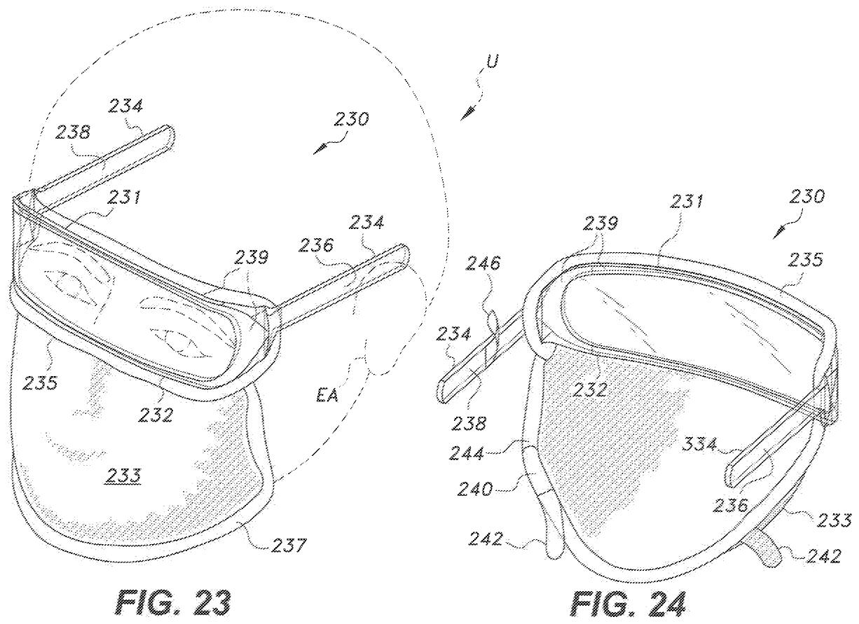

A ninth embodiment of the adhesively attached eye mask with integral face mask, designated generally as 230, is shown in FIGS. 23-24. The eye mask 230 includes a transparent (or partially light blocking) plastic eyepiece 231 having an inner peripheral surface 239 and two integral elongated transparent plastic temples or tabs 234 that are attached to the outside edges of the eyepiece 231 at their proximate ends and extend rearward therefrom. The two temples 234 include an external surface 236 away from the user's skin and an internal surface 238 adjacent to and in contact with the user's skin. Optionally, in this embodiment, the inner peripheral surface 239 of the eyepiece and the interior surface 238 of the two temples 234 that contact the user's skin are coated with an adhesive or sticky material. The adhesive provides a stronger grip by directly adhering the eyepiece 231 and the two temples 234 to the skin of the user. The eyepiece 231 may be tinted to provide UV protection in sunglass embodiments. In some embodiments, the eyepiece 231 may be mirrored or otherwise tinted so that others cannot see the eyes of the user.

The perimeter of the eyepiece 231 includes a surgical hypoallergic transparent tape or other adhesive tape 235, a portion, e.g., half, of which is attached to the perimeter of the eyepiece 231 and a portion, e.g., half, of which adheres to the face of the user U. A continuous release layer 246 covers the adhesive tape 235 to be attached to the face of the user U, and is removed prior to application. The integral face mask of the adhesively attached eye mask with integral face mask 230 includes a mask body 233, the top of which is attached to the bottom edge 232 of the eyepiece 231. In this embodiment, no ear loops are necessary. The perimeter of the mask body 233, includes a surgical hypoallergic transparent tape or other adhesive tape 237, a portion, e.g., half, of which is attached to the perimeter of the mask body 233 and a portion 244, e.g., half, of which extends beyond a lower edge of the mask body and is configured to adhere to the face of the user U. A continuous release layer 240 covers the portion 244 of the adhesive tape 237 to be attached to the face of the user U, and is removed prior to application, using one of two pull tabs 242. The adhesive tapes 235, 237 create a seal to block liquid droplets, dust, and smoke and other air pollutants from the eyes, nose, and mouth of the user U.

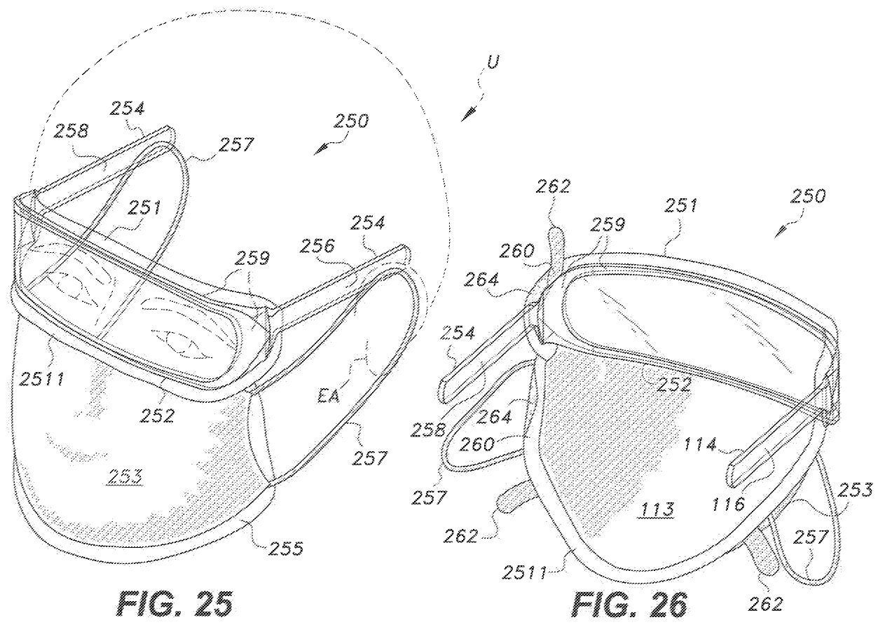

A tenth embodiment of the adhesively attached eye mask with integral face mask, designated generally as 250, is shown in FIGS. 25-26. The adhesively attached eye mask 250 in this embodiment is similar to the fourth embodiment of the adhesively attached eye mask 30 of FIGS. 3-4, described above. The eye mask 250 includes a transparent (or partially light blocking) plastic eyepiece 251 having an inner peripheral surface 259 and two integral elongated transparent plastic temples or tabs 254 that are attached to the outside edges of the eyepiece 251 at their proximate ends and extend rearward therefrom. The two temples 254 include an external surface 256 away from the user's skin and an internal surface 258 adjacent to and in contact with the user's skin. Optionally, the inner peripheral surface 259 of the eyepiece and the interior surface 258 of the two temples 254 that contact the user's skin are coated with an adhesive or sticky material. The adhesive provides a stronger grip by directly adhering the eyepiece 251 and the two temples 254 to the skin of the user. The eyepiece 251 may be tinted to provide UV protection in sunglass embodiments. In some embodiments, the eyepiece 251 may be mirrored or otherwise tinted so that others cannot see the eyes of the user. The integral face mask of the adhesively attached eye mask with integral face mask 250 includes a body 253, the top of which is attached to the bottom edge 252 of the eyepiece 251, and two ear loops 257 that are attached to the sides of the body 252 and extend rearward for attaching the face mask to the ears EA of a user U.

Also, in this embodiment, the perimeter of the eyepiece 251 includes a surgical hypoallergic transparent tape or other adhesive tape 2511, a portion, e.g., half, of which is attached to the perimeter of the eyepiece 251 and a portion, e.g., half, of which extends beyond a lower edge of the body and is configured to adhere to the face of the user U. A continuous release layer 262 covers the interior surface 258 of the two temples 254 and the portion of the adhesive tape 2511 to be attached to the face of the user U and is removed prior to application. The integral face mask of the adhesively attached eye mask with integral face mask 250 includes a mask body 253, a top edge of which is attached to the bottom edge 252 of the eyepiece 251 and two ear loops 257 that are attached to the sides of the body 253 and extend rearward for attaching the face mask to the ears EA of a user U. The perimeter of the mask body 253, includes a surgical hypoallergic transparent tape or other adhesive tape 255, a portion, e.g., half, of which is attached to the perimeter of the mask body 253 and a portion 264, e.g., half, of which adheres to the face of the user U. A continuous release layer 260 covers the portion 264 of the adhesive tape 255 to be attached to the face of the user U, and is removed prior to application, using one of two pull tabs 262. The adhesive tapes 2511, 255 create a seal to block liquid droplets, dust, and smoke and other air pollutants from the eyes, nose, and mouth of the user U.

It should be noted that any of the above face mask bodies 93, 103, 113, 132, 153, 163, 193, 212, 233, 253 may include a filter or the masks may be without a filter 97, as shown and described with respect to face mask body 93 in FIG. 9.

It is to be understood that the adhesively attached eye masks are not limited to the specific embodiments described above but encompass any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. An adhesively attached eye mask, comprising:
a transparent eyepiece having a peripheral edge including an adhesive material; and
an elongated temple attached to respective side edges of the eyepiece, each temple including an adhesive material.

2. The adhesively attached eye mask according to claim 1, wherein an inner peripheral surface of the eyepiece and an inner surface of each elongated temple is coated with the adhesive material.

3. The adhesively attached eye mask according to claim 1, wherein the adhesive material is provided on an adhesive strip positioned along a peripheral edge of the eyepiece, with a portion of the strip adhered to the peripheral edge of the eyepiece and a remaining portion of the strip extending beyond the edge of the eyepiece.

4. The adhesively attached eye mask according to claim 1, wherein each of the temples includes an ear loop at a distal end thereof.

5. The adhesively attached eye mask according to claim 1, further comprising:
a face mask extending from a bottom edge of said eyepiece, the face mask having a body including at least one layer of flexible sheet material, the body configured for covering a user's nose and mouth.

6. The adhesively attached eye mask according to claim 5, wherein an adhesive is provided along a peripheral edge of the mask body.

7. The adhesively attached eye mask according to claim 6, wherein an inner surface of the peripheral edge is coated with the adhesive.

8. The adhesively attached eye mask according to claim 7, further comprising an ear loop extending from each of opposing sides of the body of the face mask.

9. The adhesively attached eye mask according to claim 7, wherein a distal end of each of the temples includes an ear loop.

10. The adhesively attached eye mask according to claim 6, wherein the adhesive is provided on an adhesive strip positioned along the peripheral edge of the face mask.

11. The adhesively attached eye mask according to claim 10, wherein a portion of the strip is adhered to the edge of the face mask and a remaining portion of the strip extends beyond the edge of the face mask.

12. The adhesively attached eye mask according to claim 11, further comprising an ear loop extending from each of opposing sides of the body of the face mask.

13. The adhesively attached eye mask according to claim 11, wherein a distal end of each of the temples includes an ear loop.

14. The adhesively attached eye mask according to claim 5, further comprising an ear loop extending from each of opposing sides of the body of the face mask.

15. An adhesively attached eye mask, comprising:
a transparent eyepiece having a peripheral edge including an adhesive material and an elongated temple attached to respective side edges of the eyepiece, each elongated temple including an adhesive material; and
a face mask extending from a bottom edge of said eyepiece, the face mask having a body including at least one layer of flexible sheet material, the body configured for covering a user's nose and mouth.

16. The adhesively attached eye mask according to claim 15, wherein an inner peripheral surface of the eyepiece and an inner surface of each elongated temple is coated with the adhesive material.

17. The adhesively attached eye mask according to claim 15, wherein the adhesive material is provided on an adhesive strip positioned along a peripheral edge of the eyepiece, with a portion of the strip adhered to the edge of the eyepiece and a remaining portion of the strip extending beyond the edge of the eyepiece.

18. The adhesively attached eye mask according to claim 15, wherein each of the temples includes an ear loop at a distal end thereof.

19. The adhesively attached eye mask according to claim 15, further comprising an ear loop extending from each of opposing sides of the body of the face mask.
